(19) United States
(12) Patent Application Publication
(10) Pub. No.: US 2021/0038869 A1
(43) Pub. Date: Feb. 11, 2021

(54) CUTTING BALLOON AND BALLOON CATHETER

(71) Applicant: DK MEDICAL TECHNOLOGY CO., LTD., Jiangsu (CN)

(72) Inventors: Yulin WENG, Jiangsu (CN); Dongzi NIU, Jiangsu (CN); Baorui LIU, Jiangsu (CN); Quan SHI, Jiangsu (CN)

(21) Appl. No.: 16/964,223

(22) PCT Filed: Jan. 10, 2019
(86) PCT No.: PCT/CN2019/071124

§ 371 Date: Jul. 23, 2020

(30) Foreign Application Priority Data
May 18, 2018 (CN) 201810478242.X

(51) Int. Cl. A61M 25/10; A61M 25/09; A61M 25/00

(52) U.S. Cl. CPC A61M 25/1002; A61M 25/09; A61M 2025/1004; A61M 2025/105; A61M 2025/1093; A61M 25/0026

(57) ABSTRACT

A cutting balloon, comprising: a balloon body; cutting wires radially provided along the outer surface of the balloon body; two ends of each cutting wire are respectively fixed at two ends of the balloon body; the cutting wires being of a foldable structure. The surface of the cutting balloon is provided with the flexible cutting wires, so that the cutting balloon has good trafficability, and can cut off plaque tissues during expansion and reduce intimal injury; moreover, the cutting wires are fixed on the surface of the cutting balloon or can slide along a telescoping direction, thereby avoiding the problem of displacement or winding in the cutting process; furthermore, the arrangement of the foldable structure can provide axial-length compensation for the cutting wires during expansion of the balloon, and can also improve the friction between the balloon body and vascular walls and ensure the balloon not to be displaced

### CROSS REFERENCE TO RELATED APPLICATIONS

This application claims the priority to Chinese Patent Application No. 201810478242X titled “CUTTING BALLOON AND BALLOON CATHETER”, filed with the Chinese State Intellectual Property Office on May 18, 2018, which is incorporated herein by reference in its entirety.

### FIELD

The present disclosure relates to the technical field of balloon, specifically to a cutting balloon and a balloon catheter.

### BACKGROUND

Percutaneous coronary intervention (PCI) is an important method of revascularization for coronary heart disease. The number of annual coronary heart disease patients undergoing PCI in China also increases year by year. The technical update of interventional instruments and the improvement of surgical techniques have benefited more patients with coronary heart disease. The instruments that need to be used in PCI include guiding catheters, guiding guidewires, balloons, stents, and etc. The guiding guidewire is a passage provided through the coronary artery lesions of the instrument during the PCI, and is one of the important factors determining the success of PCI. The balloon is a main tool for the inflation of coronary lesions, the relief of vascular stenosis and ensuring the successful placement of the stent.

Indicators that reflect the overall performance of the balloon include balloon outer diameter, traceability, pushability, and compliance. According to different roles of the balloon in PCI, it can be divided into a pre-inflation balloon and a post-inflation balloon; and according to different compliance of the balloon, it is divided into a compliant balloon, a semi-compliant balloon, and a non-compliant balloon. Wherein, the compliant balloon has a significantly increased diameter with increasing inflation pressure and is not used in PCI; the non-compliant balloon has a small change in diameter with increasing inflation pressure, and has a higher burst pressure, and the non-compliant balloon is often configured to use for post-inflation and hard lesions after stent implantation and pre-inflation of lesions such as in-stent restenosis; the trend of the diameter of the semi-compliant balloon increasing with the inflation pressure is between the compliant and non-compliant balloons for pre-inflation of the lesion.

Pre-inflation is a preparation for lesions prior to delivery of the stent, facilitating the opening of the lumen of the delivery stent and evaluating the characteristics of the lesion. A semi-compliant balloon is usually chosen to be used for pre-inflation, and a non-compliant balloon can also be used for calcified lesions. The selected balloon should have the same length as the target lesion, and the balloon diameter can be lower than the reference blood vessel diameter. The size of the balloon is selected according to the principle of a balloon/vessel ratio of 1:1; and a nominal pressure is generally used for inflation to gradually increase pressure and inflate the balloon until the balloon no longer has a notch or has reached the rated burst pressure in the X-ray image. A balloon with a small outer diameter, a strong push force, and a short length is often used for chronic total occlusion of the coronary arteries (CTO) to perform pre-inflation, and then a large-diameter balloon is used for full pre-inflation.

Post-inflation is beneficial to ensure complete inflation and adherence of the stent, reduce stent thrombosis and late lumen loss, and reduce the target vessel/lesion revascularization rate. The post-inflation usually adopts a non-compliant balloon, the diameter of the selected post-inflation balloon should match the diameter of the stent and the reference vessel. Generally, the ratio of the diameter of the balloon: the diameter of the blood vessel is 1.1-1.2:1. The length of the post-inflation balloon should be less than the length of the stent to ensure that the balloon is located within the stent to avoid sandwiching the edge of the stent. Post-inflation should be performed for that stents with >30% residual stenosis (with “waist”), stent is not fully inflated, less than the reference vessel diameter or IVUS indicating MSD<90% reference vessel diameter, stent malapposition; and post-inflation is required for long lesions, calcified lesions, in-stent restenosis lesions, multiple stent overlaps, and the like.

Drug-eluting balloon (DEB) is a new type of balloon that has emerged in recent years, that is, the coating drugs such as paclitaxel and rapamycin that control cell proliferation are placed in the wrinkles of the balloon. When the balloon is inflated, the drug can be rapidly transported from the balloon to the wall of the blood vessel. The coating drug lost only 6% before it is delivered to the coronary circulatory system, and approximately 80% of the drug is rapidly transferred from the balloon to the vessel wall after inflation. Drug-eluting stents (DES) inhibit vascular inflammatory response and endothelial cell proliferation by coating drugs such as paclitaxel and rapamycin, thereby reducing the incidence of in-stent restenosis. But the imbalance of drug release at the junction of the stent bone and the skeleton and the stimulation of the blood vessel wall by the carrier polymer after drug depletion can increase the rate of restenosis in the stent. Compared with DES, DEB avoids the imbalance of drug release due to the absence of metal skeleton, which can evenly distribute the drug in specific vessel wall area, and preserve the original anatomy of blood vessels at the same time, and avoid the effects on blood flow patterns when treating small vessel lesions and bifurcation lesions. In the treatment of stent stenosis, double-layer stents are avoided to reduce the lumen of the vessel, and DEB has no polymer carrier, which can reduce chronic inflammation reaction and the formation of late thrombosis.

However, before the drug balloon inflates the blood vessel, it is desirable that the blood vessel lumen is fully opened, but the simple drug balloon cannot inflate the calcified or fibrotic blood vessel, and may cause secondary damage to the blood vessel.

The cutting balloon can inflate the lesion at a lower pressure, and is used in highly calcified or fibrotic intravascular lesion areas. A common cutting balloon or surface-adhesive blade forms a blade-cutting balloon, or a balloon with fixed cutting wire such as a dual-guidewire balloon is selected.

A blade-cutting balloon is a device that organically combines a conventional balloon with a microsurgical blade. When the cutting balloon is inflated, the sharp blade is exposed, and the atherosclerotic plaque and vessel wall are cut along the longitudinal direction of the vessel wall to relieve the annular pressure, and the target lesion can be maximally expanded with minimal force and time. However, the blade-cutting balloon has a poor overall permeability and is prone to excessively cutting the intima of the blood vessel due to the surface sticking to the blade.

A dual-guidewire balloon such as a safecut, a minirail, or the like is attached with a steel wire outside the ordinary balloon. When the balloon is inflated, the steel wire acts like a “blade”, but has a small outer diameter and strong ability to pass through the lesion, which is suitable for calcified lesions, in-stent restenosis (ISR) lesions, etc. But its cutting effect cannot meet expectation, and it should be gradually pressurized when the balloon is inflated, such as adding pressure for a few seconds after adding 2 atmospheres, in case the pressure is too fast, causing the two steel wires to be entangled.

Chinese Patent Application No. CN201410182654.0 discloses a single guidewire cutting balloon catheter, one end of the cutting guidewire is fixed on the distal outer wall of the balloon, and the opposite end extends into the orientation catheter. After the balloon is inflated, the single cutting guidewire cuts the lesion plaque into a single slit, and then the plaque is melted by the action of the follow-up drug, but there is little effect on the highly calcified lesion.

Chinese Patent Application No. CN201310135128.4 discloses a cutting balloon inflation catheter carrying a drug on the balloon surface, and the cutting balloon inflation catheter comprises a cutting balloon catheter body and a drug eluting, wherein the cutting balloon catheter body comprises a balloon, the surface of the balloon is provided with a plurality of blades, and the surfaces of the balloon and the blade are coated with a drug eluting. But the balloon may cause damage to the intima during the inflation and cutting of the balloon, the permeability of the blade is poor, and the permeability of the overall system is poor.

Chinese Patent Application No. CN201610266377.0 discloses a balloon catheter, comprising a needle hub, a proximal catheter, a distal catheter, a balloon, and a catheter tip, which are connected in order. The balloon catheter further comprises two intrinsic guidewires and one guiding guidewire, the balloon catheter uses the guiding guidewire as an intrinsic guidewire to reduce the number of intrinsic guidewires. The two intrinsic guidewires and the guiding guidewire for guiding are mutually cooperated to cut. Due to only one end of the guiding guidewire is fixed, it may shift in the process, which may not achieve the desired effect, especially the guiding guidewire and the intrinsic guidewire of a longer balloon are easily shifted or entangled during the cutting process.

### SUMMARY

In view of this, the technical problem to be solved in the present disclosure is to provide a cutting balloon and a balloon catheter, and the cutting balloon has better permeability and is not easily displaced during a cutting process.

A cutting balloon is provided according to the present disclosure, comprising:

a balloon body;

a cutting wire fixed longitudinally on an outer surface of the balloon body;

two ends of the cutting wire are respectively fixed to two ends of the balloon body;

and the cutting wire is foldable.

A cutting balloon is further provided according to the present disclosure, comprising:

a balloon body;

a cutting wire fixed longitudinally on an outer surface of the balloon body; the cutting wire is foldable; and an orientation groove is provided on an outer surface of one end of the balloon body; one end of the cutting wire is fixed to the orientation groove, and the opposite end of the cutting wire is fixed to the opposite end of the balloon body

A cutting balloon is further provided according to the present disclosure, comprising:

a balloon body;

a cutting wire fixed longitudinally on an outer surface of the balloon body; the cutting wire is foldable;

a first orientation groove is provided on the outer surface of one end of the balloon body, a second orientation groove is provided on the outer surface of the opposite end of the balloon body; and one end of the cutting wire is fixed to the first orientation groove, and the opposite end is fixed to the second orientation groove.

Preferably, a length of the cutting wire is 1-100 mm longer than the length of the balloon body in the deflated state.

Preferably, the number of the cutting wire is n, and n is an integer greater than or equal to 2; and the angle between any two of the cutting wires is 360°/n.

Preferably, the cutting wire is in a foldable wave structure and/or a helical spring structure.

Preferably, the cross section of the helical spring structure is one or more selected from a circle, a triangle, and a rectangle.

Preferably, wherein one or two ends of the cutting wire close to the end of inflated balloon body is in the foldable structure, and the remaining portion is in a linear structure.

Preferably, the outer surface of the balloon body is further provided with a drug eluting.

A balloon catheter is further provided in the present disclosure, comprising a distal tube, the cutting balloon and a catheter in order;

the distal tube is not in communication with the cutting balloon; the cutting balloon is in communication with the catheter;

an inner tube is provided in the cutting balloon; one end of the inner tube is in communication with the distal tube;

when the catheter is a multi-lumen tube, the opposite end of the inner tube is in communication with one lumen of the multi-lumen tube;

when the catheter is a single-lumen tube, the opposite end of the inner tube extends through the catheter; and

a guiding guidewire is through the distal tube, the inner tube, and the catheter.

A cutting balloon is provided according to the present disclosure, comprising: a balloon body; a cutting wire fixed longitudinally on the outer surface of the balloon body; two ends of the cutting wire are respectively fixed to two ends of the balloon body; and the cutting wire is foldable. Compared with the conventional art, a flexible cutting wire is fixed to the surface of the cutting balloon according to the present disclosure, which has better permeability, and can cut a plaque tissue when dilating, and has less damage to an intima. At the same time, the cutting wire is fixed on the surface of the cutting balloon, or can slide in a direction of extension and retraction, thereby avoiding problems of shifting and entangling. Moreover, the foldable structure can not only provide axial length compensation for the cutting wire when the balloon is inflated, but also increase the friction between the balloon body and the blood vessel wall to ensure that the balloon does not shift.

### BRIEF DESCRIPTION OF DRAWINGS

FIG. 1 is a schematic view showing the structure of a cutting wire with a circular helical section provided by the present disclosure.

FIG. 2 is a schematic view showing the structure of a cutting wire with a triangular helical section provided by the present disclosure.

FIG. 3 is a schematic view showing the cross-section of a cutting balloon provided by the present disclosure (wherein the figure on the left is a dual-cutting wire, and the figure on the right is a three-cutting wire).

FIG. 4 is a schematic view showing the structure of a balloon catheter placed at vascular lesion sites in unexpanded state provided by the present disclosure.

FIG. 5 is a schematic view showing the structure of a balloon catheter placed at vascular lesion sites in fully inflated state provided by the present disclosure.

FIG. 6 is a schematic view showing the structure of a balloon catheter placed at vascular lesion sites after inflation provided by the present disclosure.

FIG. 7 is a schematic view showing the structure of the balloon catheter according to Example 1 of the present disclosure.

FIG. 8 is a schematic view showing the cross-sectional structure of the catheter used in Example 1 of the present disclosure.

FIG. 9 is a schematic view showing the structure of the balloon catheter provided by Example 2 of the present disclosure.

### DETAILED DESCRIPTION

The technical solutions in the embodiments of the present disclosure are clearly and completely described in the following with reference to the embodiments of the present disclosure. Apparently, the described embodiments are only a part of the embodiments of the present disclosure, and not all of the embodiments. All other embodiments obtained by one of ordinary skill in the art based on the embodiments of the present disclosure without creative efforts fall within the scope of protection of the present disclosure.

A cutting balloon is provided according to the present disclosure, comprising:

a balloon body;

a cutting wire fixed longitudinally on an outer surface of the balloon body;

two ends of the cutting wire are respectively fixed to two ends of the balloon body;

and the cutting wire is foldable.

According to the present disclosure, the balloon body is preferably a nylon balloon body or a polyether block amide balloon body. The balloon body is preferably produced by a following method: extruding a nylon (PA) or a polyether block amide (PEBAX) to produce a single-lumen tube as a raw material of a balloon body, and then subjecting the raw material to hot blow molded on a balloon former containing a mold with protrusions.

The outer surface of the balloon body is disposed with a cutting wire in a longitudinal direction; the cutting wire is preferably located in a foldable wing of a balloon body in deflated state; the diameter of the cutting wire is preferably 0.1-0.035 inches. The number of the cutting wires is preferably n; n is an integer greater than or equal to 2, more preferably an integer of 2-8, and further preferably an integer of 2-6. The angles between any two of the cutting wires are preferably 360°/n, which are distributed circumferentially along the balloon body. A plurality of cutting wires can regularly cut the plaque tissue when the cutting balloon is inflated, and the damage to the intima is small. At the same time, the balloon body has a certain supporting effect after being inflated, and increases the friction between the balloon body and the blood vessel wall to ensure that the balloon is not shifted. The length of the cutting wire varies with different specifications of the balloon, and it is preferable that the length of the cutting wire is 1-100 mm longer than the length of the balloon body in the deflated state the present disclosure. The material of the cutting wire may be any material well known to one of ordinary skill in the art, and is not particularly limited, and may be medical stainless steel, cobalt chromium alloy, nickel titanium alloy or other metal or alloy with better biocompatibility, or may be a polymer material with good biocompatibility such as polyamide or polyethylene.

The two ends of the cutting wire are fixed to the two ends of the balloon body; the other parts of the cutting wire may be partially fixed to the outer surface of the balloon body or may not be fixed, and there is no special limitation; the method well known to one of ordinary skill in the art is not particularly limited, and is preferably hot melt, adhesive or mechanically fixed in the present disclosure.

Or a first orientation groove is provided on an outer surface of one end of the balloon body, and a second orientation groove is provided on the outer surface of the opposite end; one end of the cutting wire is fixed to the first orientation groove, and the opposite end is fixed to the second orientation groove so that both ends can only move along the orientation groove.

Or an orientation groove is provided on the outer surface of one end of the balloon body, one end of the cutting wire is fixed to the orientation groove, and the opposite end is fixed to the opposite end of the balloon body; the cutting wire can slide along the orientation groove.

One or two ends of the cutting wire are fixed on the surface of the balloon body or fixed to the orientation groove to avoid the problem of shifting and entangling of the cutting wire when the balloon is inflated.

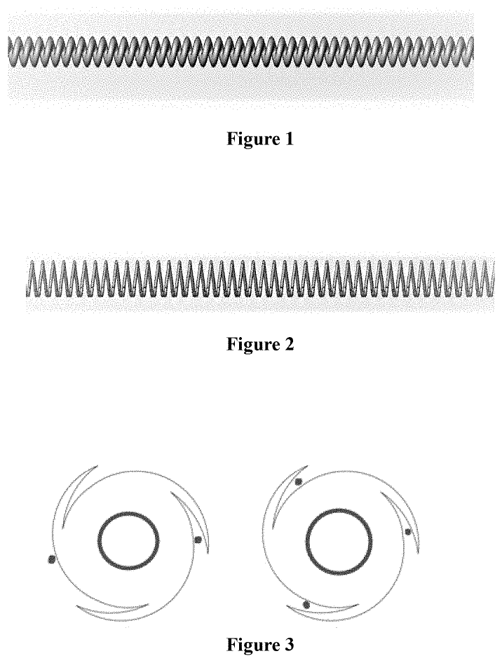

In the present disclosure, the cutting wire comprises a foldable structure. On the one hand, it can provide axial length compensation for the cutting wire when the balloon is inflated; and on the other hand can increase the friction between the balloon and the vascular wall to ensure that the balloon does not shift. The foldable structure may be a foldable structure well known to one of ordinary skill in the art, and is not particularly limited, a foldable wave structure and/or a helical spring structure are preferred in the present disclosure. When the foldable structure is a foldable wave structure, it forms wrinkles, and the width of the wrinkles is preferably 0.1-0.55 mm. When the foldable structure is a helical spring structure, the cross-section of the helical spring structure of the present disclosure is not particularly limited, and may be any shape that is advantageous for cutting, preferably one or more selected from a circle, a triangle, and a rectangle. The side length of the triangular cross-section is preferably 0.1-0.55 mm, and the height is preferably 0.1-0.5 mm; the diameter of the circular cross-section is preferably 0.2-0.5 mm; and the side length of the rectangular cross-section is preferably 0.2-0.5 mm. When the cross-section of the helical spring structure is a triangle and a rectangle, the radii of chamfering of the helical structure are each independently preferably 0.01 to 0.05 mm. The cutting wire may be a foldable structure as a whole or a foldable structure in partial, and is not particularly limited. In the present disclosure, it is preferable that the whole is a foldable structure, or one or two ends of the cutting wire close to the end of inflated balloon body is in the foldable structure, and the remaining portion is in a linear structure. The length of the foldable structure of the cutting wire is preferably 5-300 mm. With reference to FIGS. 1, 2 and 3, FIG. 1 is a schematic view showing the structure of a cutting wire with a circular helical section provided by the present disclosure; FIG. 2 is a schematic view showing the structure of a cutting wire with a triangular helical section provided by the present disclosure; and FIG. 3 is a schematic view showing the cross-section of a cutting balloon provided by the present disclosure wherein the figure on the left is a dual-cutting wire, the figure on the right is a three-cutting wire, the black point is a cross-section of the cutting wire, and the black ring is an inner tube section.

During the inflation process of the balloon body, the foldable structure of the cutting wire is configured to axially elongate the cutting wire when the balloon body is inflated, so that the balloon body is not deformed by stretching when the cutting wire is deformed; when the balloon body is restored to its original shape, the foldable structure of the cutting wire is partially shrunk, so that the cutting wire can be linearly covered on the surface of the balloon body to facilitate the integrally withdrawal from the body.

According to the present disclosure, the outer surface of the balloon body is preferably further provided with a drug eluting. At this time, the cutting balloon can both cut the plaque tissue and transfer the drug on the surface of the balloon to the surface of the blood vessel, thereby effectively inhibiting the excessive intimal hyperplasia of the damaged blood vessel.

Flexible cutting wires are fixed to the surface of the cutting balloon according to the present disclosure, which has better permeability, and can cut a plaque tissue when dilating, and has less damage to an intima. At the same time, the cutting wires are fixed on the surface of the cutting balloon, or can slide in a direction of extension and retraction, thereby avoiding problems of shifting and entangling during cutting. Moreover, the foldable structure can not only provide axial length compensation for the cutting wire when the balloon is inflated, but also increase the friction between the balloon body and the blood vessel wall to ensure that the balloon does not shift.

A balloon catheter is further provided according to the present disclosure, comprising a distal tube, a cutting balloon and a catheter in order; the distal tube is not in communication with the cutting balloon; the cutting balloon is in communication with the catheter; an inner tube is provided in the cutting balloon; one end of the inner tube is in communication with the distal tube; when the catheter comprises a multi-lumen tube, the opposite end of the inner tube is in communication with one lumen of the multi-lumen tube; when the catheter is a single-lumen tube, the opposite end of the inner tube extends through the catheter; and a guiding guidewire passes through the distal tube, the inner tube, and the catheter.

The guiding guidewire is penetrated from the distal tube through the inner tube in the cutting balloon to reach the proximal end of the cutting balloon and then enters to the catheter. The distal tube is the distal tube well known to one of ordinary skill in the art and is not particularly limited, and the inner diameter according to the present disclosure is preferably not less than 0.3 mm; more preferably, the inner diameter thereof can pass through three specifications of guiding guidewires of 0.014 inches, 0.018 inches and 0.035 inches. The inner tube is the inner tube well known to one of ordinary skill in the art and is not particularly limited, and the inner diameter according to the present disclosure is preferably not less than 0.3 mm; more preferably, the inner diameter thereof can pass through three specifications of guiding guidewires of 0.014 inches, 0.018 inches and 0.035 inches. The present disclosure is preferable that one end of the inner tube in communication with the distal tube is connected with the cutting balloon to form a balloon cavity sealing point. Both ends of the inner tube are preferably provided with a developing unit, and more preferably a developing unit is fixed to at an inner tube position opposite to the highest point of the slope portion when the cutting balloon is in the inflation state. The developing unit is the developing unit known to one of ordinary skill in the art, and is not particularly limited. In the present disclosure, it is preferably a thin-walled annular body made from a platinum alloy or other metal and plastic which are impervious to X-ray material. The developing unit is visible under X-rays, and the position of the balloon cutting working area can be identified during the operation, so that the operator can smoothly deliver the balloon to the lesioned blood vessel area. The catheter is a catheter known to one of ordinary skill in the art and is not particularly limited, and may be a multi-lumen tube, a single-lumen tube or a multi-lumen tube connected to a single lumen tube. When the catheter is a single-lumen tube, the catheter is in communication with the cutting balloon at the same time, and the inner tube is preferably located inside the lumen of the catheter. The inner tube and the catheter form a coaxial setting or multi-lumen configuration, and the guiding guidewire is inside the inner tube. When the catheter is a multi-lumen tube, preferably one of the lumens is in communication with the inner tube, and the other lumens are in communication with the cutting balloon; and the lumen in communication with the inner tube is for passing through the guiding guidewire, and the inner diameter thereof is preferably not less than 0.3 mm, and more preferably, the inner diameter thereof is passed through guiding guidewires of three specifications of 0.014 inches, 0.018 inches, and 0.035 inches. The cutting balloon is the same as described above, and is not described herein again; the material of the catheter is preferably polyether block amide (PEBAX).

According to the present disclosure, the balloon catheter can be an integral exchange type balloon catheter or a rapid exchange type balloon catheter.

When the balloon catheter is an integral exchange type balloon, the end of the catheter away from the cutting balloon is connected with a Y-shaped connecting component, the Y-shaped connecting component comprises a guiding guidewire outlet and a balloon filling port. The Y-shaped connecting component cooperates with the catheter to construct two lumens, that is, a balloon airway for inflating and deflating the balloon and a multifunctional lumen passed through by the guiding guidewire. When the catheter is a single-lumen tube, the balloon filling port of the Y-shaped connecting component is in communication with the cutting balloon through the catheter to form a balloon airway, and the inner tube through which the guiding guidewire passes is in communication with the outlet of the guiding guidewire, forming a multifunctional lumen. When the catheter is a multi-lumen tube, the lumen through which the guiding guidewire passes is in communication with the outlet of the guiding guidewire, forming a multifunctional lumen. The balloon filling port is in communication with the cutting balloon through other lumens to form a balloon airway. In addition to being used for the passing of the guiding guidewire, the multifunctional lumen can also be used for injecting heparin, contrast agents and the like.

When the balloon catheter is a rapid exchange type balloon catheter, the catheter at the proximal end of the balloon catheter is provided with a rapid exchange guidewire port, and the catheter is in communication with the balloon filling port, the balloon filling port is not in communication with the rapid exchange guidewire port. The distance between the rapid exchange guidewire port and the proximal end of the balloon catheter (that is, the balloon filling port) is preferably 10 to 1000 mm. When the catheter is a single-lumen tube, the inner tube passed through which the guiding guidewire passes forms a coaxial setting or multi-lumen configuration in the catheter, and the inner tube is in communication with the rapid exchange guidewire port fixed to the catheter. When the catheter is a multi-lumen tube, the lumen passed through which the guiding guidewire passes is in communication with the rapid exchange guidewire port. The balloon filling port is in communication with an external stamping device.

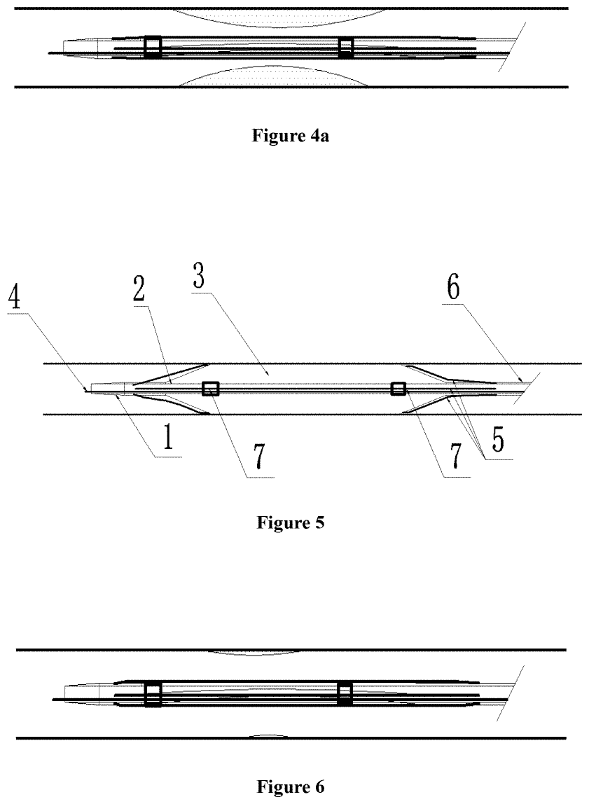

With reference to FIGS. 4,5 and 6, FIG. 4 is a schematic view showing the structure of a balloon catheter placed at vascular lesion sites in the unexpanded state of the vascular lesion provided by the present disclosure; FIG. 5 is a schematic view showing the structure of a balloon catheter placed at vascular lesion sites in the fully expanded inflated state of the vascular lesion provided by the present disclosure, wherein 1 is a distal tube, 2 is an inner tube, 3 is a balloon body, 4 is a guiding guidewire, 5 is a cutting wire, 6 is a catheter, 7 is a developing unit; and FIG. 6 is a schematic view showing the structure of a balloon catheter placed at vascular lesion sites after inflation provided by the present disclosure.

The balloon catheter provided by the present disclosure moves along the guidewire in the percutaneous lumen to the vascular lesion, in a deflation state. The cutting wires are uniformly distributed in the foldable wing outside the balloon. When the pressure inside of the balloon increases, the balloon body is inflated into a column shape by internal pressure, and the cutting wire becomes fusiform with the inflation of the balloon body, and the cutting wire contacts the plaque tissue at the lesioned blood vessel and cuts to tear the plaque tissue. After repeatedly filling the balloon for several times, when the plaque tissue is fully cut, the balloon is suctioned under negative pressure to recover the balloon body to the original state, the cutting wire linearly covers the surface of the balloon body as the balloon inflation pressure disappears.

In order to further illustrate the present disclosure, a cutting balloon and a balloon catheter provided by the present disclosure will be described in detail below with reference to the examples.

The reagents used in the following examples are all commercially available.

### Example 1

A balloon catheter is provided as shown in FIG. 7, wherein 1 is a distal tube, 2 is an inner tube, 3 is a balloon body, 4 is a guiding guidewire, 5 is a cutting wire, 6 is a catheter, and 7 is a developing ring, 8 is a Y-shaped connecting component, 9 is a balloon filling port, and 10 is a guiding guidewire outlet.

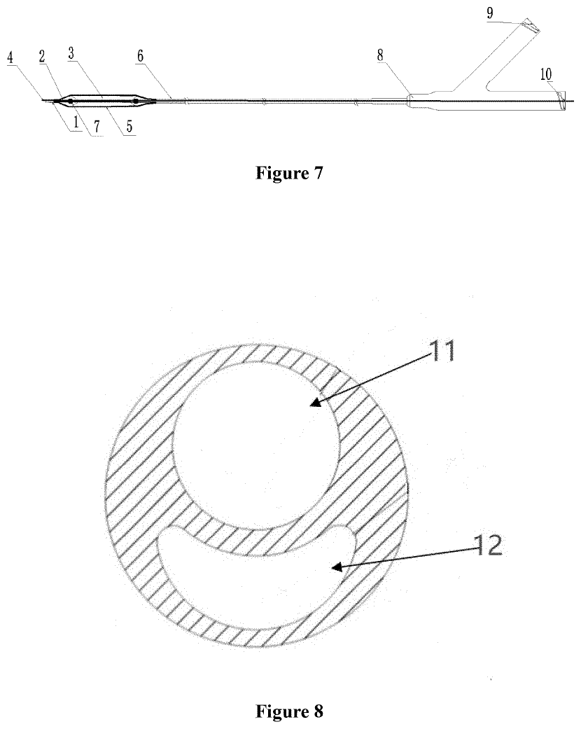

As shown in FIG. 7, a balloon 3 is connected to the catheter 6 by means of laser welding or the like. The catheter 6 is a double-lumen tube of polyether block amide (PEBAX) material, and the cross-sectional structure thereof is shown in FIG. 8. The first lumen 11 thereof is circular and is used for the guiding guidewire to pass through, which is in conduction with the inner tube 2 and has an inner diameter of not less than 0.30 mm, and further preferably has an inner diameter through which a guiding guidewire of three specification of at least 0.014 inches, 0.018 inches, and 0.035 inches is able to pass through. The outer cutting wires 5 of the balloon body 3 are uniformly distributed in a circumferential shape, the angle of the dual-cutting wire is 180 degrees, the angle of the three-cutting wire is 120 degrees, and so on, wherein the cutting wire is 1-100 mm slightly longer than the balloon body 3. The developing rings 7 are two thin-walled annular bodies made of platinum-iridium alloys, which are respectively fixed at two ends of the inner tube 2 fixed to the balloon body 3, and the developing ring is visible under X-rays. The position of the cutting wire 5 working area can be identified during the operation, so that the operator can smoothly deliver the balloon to the lesioned blood vessel area.

Also, as shown in FIG. 7, the free end of the catheter is connected to a Y-shaped connecting component 8, and the Y-shaped connecting component 8 cooperates with the catheter 6 to form a balloon airway for inflating and deflating the balloon and a multifunctional lumen passed through by the guiding guidewire 4, that is, the balloon airway is formed by a combination of the second lumen of the catheter 6 and one lumen of the Y-shaped connecting component 8. The multifunctional lumen is formed by splicing the first lumen of the catheter 6 and the opposite lumen of the Y-shaped connecting component 8, and the multifunctional lumen can be used for injecting heparin, contrast agent, etc., in addition to allow the guidewire 4 to pass through. The two ports of the Y-shaped connecting component 8 are all Luer, preferably 6% standard Luer.

Clinical specific embodiment: preoperative angiography is used to evaluate the condition of the lesion blood vessel, and a suitable size of the balloon catheter and catheter sheath are selected. A suitable site to puncture the blood vessel is selected, and the guidewire is placed into the vascular lesion site through the catheter sheath. The proximal end of the guidewire is inserted along the tip end hole of the balloon catheter, and the balloon catheter is pushed forward until to the vascular stenosis site. The guidewire can be withdrawn if necessary, and a contrast agent is injected from the guidewire outlet of the Y-shaped connecting component to observe the lesion site. After the angiography is completed, the guidewire is reinserted, the pump is connected to the balloon filling port of the Y-shaped connecting component, the pump is started until the pressure is larger than the nominal pressure of the balloon. After 5-240 seconds, the inflation of the balloon is completed, and the multi-cutting wires around the balloon are squeezed to the vessel wall and cut the lesioned tissue. The pump is started to retract the balloon completely, and then the balloon catheter is withdrawn.

### Example 2

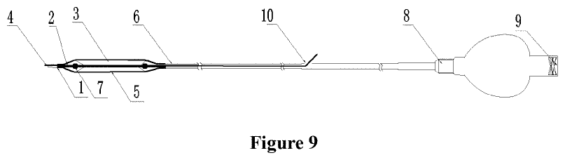

A schematic view of the balloon catheter is provided as shown in FIG. 9. This example is identical to the example 1 except for the pushing portion and the connecting component, and details are not described herein again, wherein in FIG. 9, 1 is a distal tube, 2 is an inner tube, 3 is a balloon, 4 is a guiding guidewire, 5 is a cutting wire, 6 is a catheter, 7 is a developing ring, 8 is a connecting component, and 9 is a balloon filling port, 10 is a rapid exchange guidewire port.

As shown in FIG. 9, a balloon 3 is connected to the catheter 6 by means of laser welding or the like. The distal end of the inner tube 2 passes through the interior of the balloon body 3 and is connected to balloon at the distal end of the balloon, forming a balloon cavity sealing point. The inner tube 2 allows the guiding guidewire 4 to pass through its lumen. The common nominal size of the guiding guidewire 4 may be 0.014 inches, 0.018 inches, and 0.035 inches. The inner tube 2 may be a coaxial shape or may be a multi-lumen shape inside the catheter 6. The inner tube 2 and the catheter 6 are made of commonly used medical grade plastic materials such as polyamide, polyethylene, and the like. The catheter 6 is 10-1000 mm from the proximal end of the balloon, the inner tube 2 passes through from the catheter 6, and the inner tube 2 is welded together with the catheter 6 to form a rapid exchange guidewire port 10. The proximal end of the catheter 6 is connected to the connecting component 8. The connecting component 8 is adhered to the catheter 6 to form a pressure filling lumen, and the external stamping device is in communication with the connecting component 8 through the balloon filling port 9, and the stamping pump enters the catheter 6 through the pressurizing chamber connecting component 8 until to the balloon body 3.

The clinical embodiment of the present disclosure: preoperative angiography is used to evaluate the condition of the lesion blood vessel, and a suitable size of the balloon catheter and catheter sheath are selected. A suitable site to puncture the blood vessel is selected, and the guidewire is placed into the vascular lesion site through the catheter sheath. The proximal end of the guidewire is inserted along the tip end hole of the balloon catheter, and the balloon catheter is pushed forward until to the vascular stenosis site. The pump is connected to the balloon filling port of the connecting component, the inflation pump is started until the pressure is larger than the nominal pressure of the balloon. After 5-240 seconds, the inflation of the balloon is completed, and the multi-cutting wires around the balloon are squeezed to the vessel wall and cut the lesioned tissue. The pump is started to deflate the balloon completely, and then the balloon catheter is withdrawn.

The above description is only a preferred embodiment of the present disclosure. It should be noted that a number of modifications and improvements may be made by one of ordinary skill in the art without departing from the principles of the present disclosure, and such modifications and improvements are also considered to fall within the scope of protection of the present disclosure.

1. A cutting balloon, comprising:
a balloon body;
a cutting wire fixed longitudinally on an outer surface of the balloon body;
two ends of the cutting wire are respectively fixed to two ends of the balloon body;
and
the cutting wire is foldable.

2. The cutting balloon according to claim 1, wherein
an orientation groove is provided on the outer surface of one end of the balloon body; one end of the cutting wire is fixed to the orientation groove, and the opposite end of the cutting wire is fixed to the opposite end of the balloon body.

3. The cutting balloon according to claim 2, wherein
a first orientation groove is provided on an outer surface of one end of the balloon body, a second orientation groove is provided on the outer surface of the opposite end of the balloon body; and
one end of the cutting wire is fixed to the first orientation groove, and the opposite end is fixed to the second orientation groove.

4. The cutting balloon according to claim 1, wherein the length of the cutting wire is 1-100 mm longer than the length of the balloon body in the deflated state.

5. The cutting balloon according to claim 1, wherein a number of the cutting wire is n, and n is an integer greater than or equal to 2; and the angle between any two of the cutting wires is 360°/n.

6. The cutting balloon according to claim 1, wherein the cutting wire is in a foldable wave structure and/or a helical spring structure.

7. The cutting balloon according to claim 6, wherein the cross section of the helical spring structure is one or more selected from a circle, a triangle, and a rectangle.

8. The cutting balloon according to claim 1, wherein one or two ends of the cutting wire close to the end of inflated balloon body is in the foldable structure, and the remaining portion is in a linear structure.

9. The cutting balloon according to claim 1, wherein the outer surface of the balloon body is further provided with a drug eluting.

10. A balloon catheter, comprising a distal tube, a catheter and the cutting balloon according to claim 1 in order;
the distal tube is not in communication with the cutting balloon; the cutting balloon is in communication with the catheter;
an inner tube is provided in the cutting balloon; one end of the inner tube is in communication with the distal tube;
when the catheter is a multi-lumen tube, the opposite end of the inner tube is in communication with one lumen of the multi-lumen tube;
when the catheter is a single-lumen tube, the opposite end of the inner tube extends through the catheter; and
a guiding guidewire is provided through the distal tube, the inner tube, and the catheter.
